(19) United States
(12) Patent Application Publication
(10) Pub. No.: US 2023/0219214 A1
(43) Pub. Date: Jul. 13, 2023

(54) LINK,ROBOTIC ARM AND ROBOT

(71) Applicant: Cornerstone Technology (Shenzhen) Limited, Shenzhen (CN)

(72) Inventor: Zerui Wang, Shenzhen (CN)

(21) Appl. No.: 18/187,580

(22) Filed: Mar. 21, 2023

Related U.S. Application Data
(63) Continuation of application No. PCT/CN2021/132877, filed on Nov. 24, 2021.

(30) Foreign Application Priority Data
Jan. 6, 2021 (CN) 202110013227.X

(51) Int. Cl. B25J 9/00; B25J 18/00

(52) U.S. Cl. CPC B25J 9/0015; B25J 18/00

(57) ABSTRACT

A link, a robotic arm and a surgical robot are provided. The link is configured in a rod shape and defines an inner cavity extending in a length direction of the link, and wherein at least a part of the inner cavity is seamlessly enclosed in a circumference perpendicular to the length direction of the link. The robotic arm includes at least one link. The surgical robot includes at least one robotic arm.

### CROSS-REFERANCE TO RELATED APPLICATIONS

This application is a continuation of International Application No. PCT/CN2021/132877, filed on Nov. 24, 2021, which claims priority to Chinese Patent Application No. 202110013227.X, filed on Jan. 6, 2021, both of which are hereby incorporated by reference in their entireties.

### TECHNICAL FIELD

The present disclosure relates to the technical field of machinery, in particular to a link, a robotic arm and a robot.

### BACKGROUND

Medical surgical micro-instruments are widely used in various operations due to the advantages of accurate positioning, stable operation, desirable flexibility, large working range, radiation and infection protection, and the like. The surgical robot is a type of medical equipment. In the operation, the patient on the patient's bed is pushed to a position in front of the surgical robot, and the surgical robot has its multiple robotic arms controlled to move onto the patient's body to perform the corresponding surgery via one or more preset hole of the patient's skin. During the operation, a surgical micro-instrument held by one of the robotic arms can be rotated around a respective rotation point (called RCM, Remote Center of Motion, point) at the respective preset hole and inserted into the patient via the respective preset hole.

The existing surgical robots have been developed to have a function of realizing the rotation of the robotic arm around the RCM point by the constraint of a mechanical parallelogram mechanism. For example, steel wires or straps are employed for transmission and constraint of the rotation of the links of the robotic arm, so that a rod of an effector unit connected to an end of the robotic arm exhibits a coupling characteristic of a parallelogram. In a structure of the robotic arm in which multiple links are connected in series and which is very long, the compactness and high rigidity of the robotic arm with a strap drive train have a crucial impact on the surgical performance of the surgical robot.

FIG. 1 shows an existing structure employed for a link of a robotic arm, the link includes an upper cover 11 and a lower bottom plate 12 that are connected by a fastener, an accommodating cavity is formed between the upper cover 11 and the lower bottom plate 12 to receive a flexible transmission assembly. The link of this structure requires higher rigidity of the lower bottom plate 12, especially the link at the distal end of the robotic arm which is subjected to both a large bending moment and a large torque during its movement and the operation of the medical staff. Since there is a seam at the connection between the upper cover 11 and the lower bottom plate 12, a displacement between layers occurs at the seam, and meanwhile the lower bottom plate 12 is subjected to a relatively large torsional deformation. For the elongated robotic arm including multiple links connected in series, the torsional deformations of the links accumulate to result in a large operating error of the end effector, thereby reducing the accuracy of the surgical robot.

To this end, the present disclosure provides a link, a robotic arm and a robot to at least partially solve the problems in the existing knowledge.

### SUMMARY

A series of concepts in simplified form have been introduced in the summary section, which are described in further detail in the detailed description section. The summary section of the present disclosure does not mean to attempt to limit the key features and essential technical features of the claimed technical solution, nor does it mean to attempt to determine the protection scope of the claimed technical solution.

According to a first aspect of the present disclosure, a link for a robotic arm is provided. The link defines an inner cavity extending in a length direction of the link. At least a part of the inner cavity is seamlessly enclosed in a circumference perpendicular to the length direction of the link.

According to a second aspect of the present disclosure, a robotic arm comprising at least one link is provided. The link defines an inner cavity extending in a length direction of the link. At least a part of the inner cavity is seamlessly enclosed in a circumference perpendicular to the length direction of the link.

According to a third aspect of the present disclosure, a surgical robot is provided. The surgical robot includes at least one robotic arm. The robotic arm includes at least one link. The link defines an inner cavity extending in a length direction of the link. At least a part of the inner cavity is seamlessly enclosed in a circumference perpendicular to the length direction of the link.

### BRIEF DESCRIPTION OF THE DRAWINGS

The following drawings of the present disclosure are incorporated herein as a part of the present disclosure for understanding of the present disclosure. The drawings illustrate embodiments and description of the present disclosure, which is used to explain the principle of the present disclosure.

FIG. 1 is a perspective exploded schematic view of a link of a robotic arm of a robot in the existing knowledge;

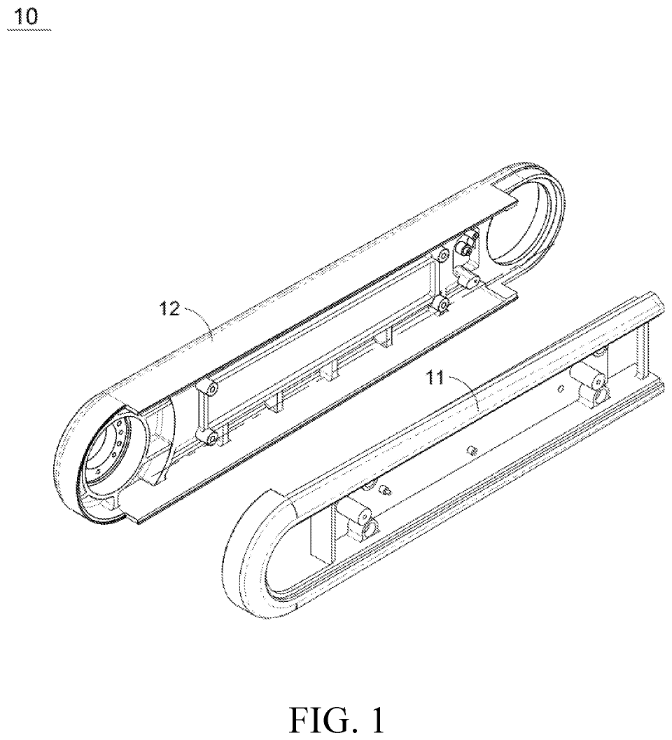

FIG. 2 is a schematic perspective view of a partial structure of a robot according to an embodiment of the present disclosure, in which a robotic arm and an end effector are shown;

FIG. 3 is a schematic perspective view of a partial structure of the robot in FIG. 2, in which a robotic arm and an end effector are shown and a partial structure of the robotic arm is shown in an exploded view;

FIG. 4 is a perspective view of a link of the robotic arm in FIG. 3;

FIG. 5 is another perspective schematic view of the link of the robotic arm in FIG. 3; and

FIG. 6 is a schematic cross-sectional view of the robotic arm in FIG. 5.

### DETAILED DESCRIPTION

In the following description, numerous specific details are set forth in order to understand the present disclosure thoroughly. It will be apparent, however, for those skilled in the art that embodiments of the present disclosure may be practiced without one or more of these details. In other examples, some technical features well-known in the art are not described in order to avoid confusion with the embodiments of the present disclosure.

For a thorough understanding of the embodiments of the present disclosure, detailed structures will be presented in the following description. Obviously, the implementation of the embodiments of the present disclosure is not limited to the specific details familiar to those skilled in the art. It should be noted that the ordinal numbers such as “first” and “second” quoted in the present disclosure are merely identifications, and do not have any other meanings, such as a specific order. Also, for example, the term “a first element” does not by itself imply the presence of “a second element”, nor does the term “a second element” by itself imply the presence of “a first element”. The terms “upper”, “lower”, “front”, “rear”, “left”, “right” and similar expressions used in the present disclosure are for the purpose of illustration and not limitation.

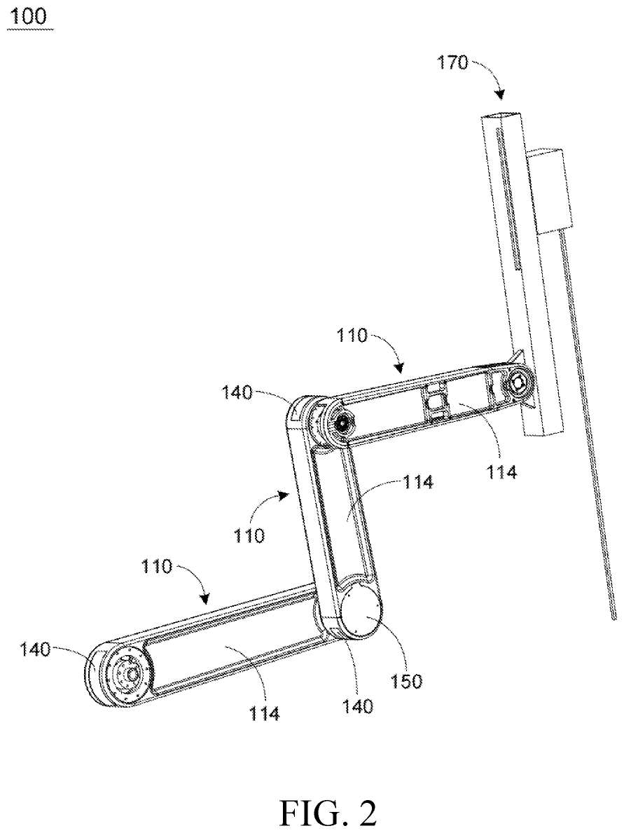

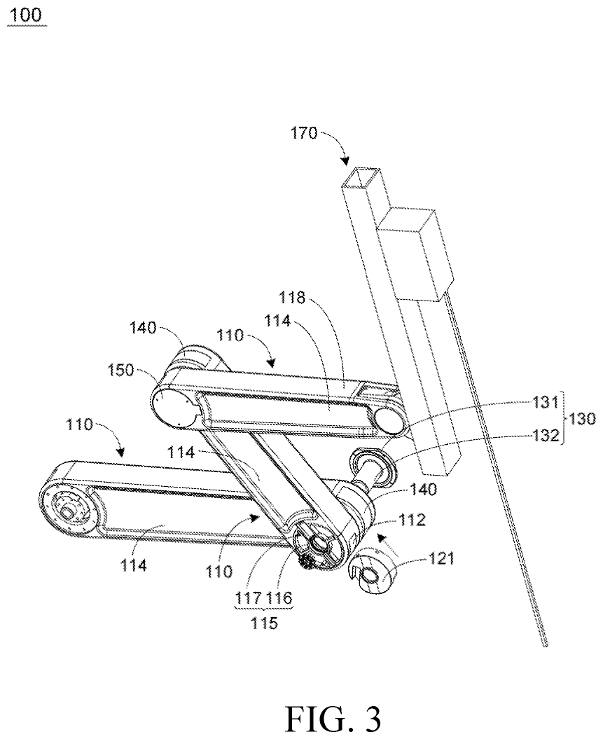

As shown in FIG. 2 and FIG. 3, the present disclosure provides a link 110, a robotic arm 100 and a robot. The robot can be a surgical robot, an industrial robot, an entertaining or teaching robot, and the like. In this embodiment, the description is made by taking a surgical robot as an example of the robot.

The robot may include at least one robotic arm 100 and an effector 170 disposed at an end of the robotic arm 100. The robotic arm 100 can drive the effector 170 to be inserted into a preset hole of the patient's skin and rotate to complete the corresponding operation. Since the effector 170 is a structure well-known in the art, it will not be described in detail.

The robotic arm 100 according to the present disclosure will be described in detail below with reference to FIG. 2 to FIG. 6.

The robotic arm 100 mainly includes at least one link 110. In the case that the robotic arm 100 includes at least two links 110, the at least two links 110 are connected end to end and two adjacent links 110 are pivotally connected. FIG. 2 and FIG. 3 exemplarily show three links 110 which are connected end to end. It can be understood that the number of the links 110 can be determined according to actual needs, and for example, it can be one, four or more.

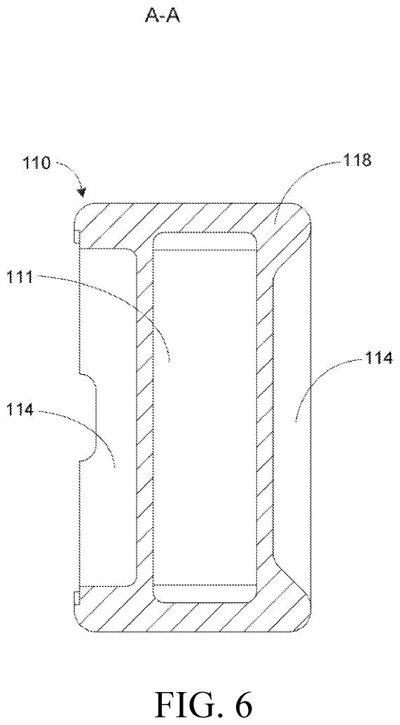

In the present embodiment, the link 110 has a length and defines an inner cavity 111 extending in a length direction L of the link 110. The inner cavity 111 extends straight from end to end of the link 110 in a direction (specifically, the length direction L of the link 110). As shown in FIG. 6, at least a part of the inner cavity 111 is seamlessly enclosed in section perpendicular to the length direction L of the link 110, so that the smooth transmission of the force flow and the continuity of the force can be ensured. In this way, the link 110 having a relative small wall thickness and a relative simple structure can meet the requirements for rigidity and torsional strength.

In the present embodiment, the cross section of the inner cavity 111 is configured in a rectangle shape, that is, the section of the inner cavity 111 perpendicular to the length direction L of the link 110 has a shape of a rectangle. Those skilled in the art can understand that the shape of the cross section of the inner cavity 111 is not limited to this embodiment and may also be a circle, an ellipse, a square, an irregular shape or any other suitable shape.

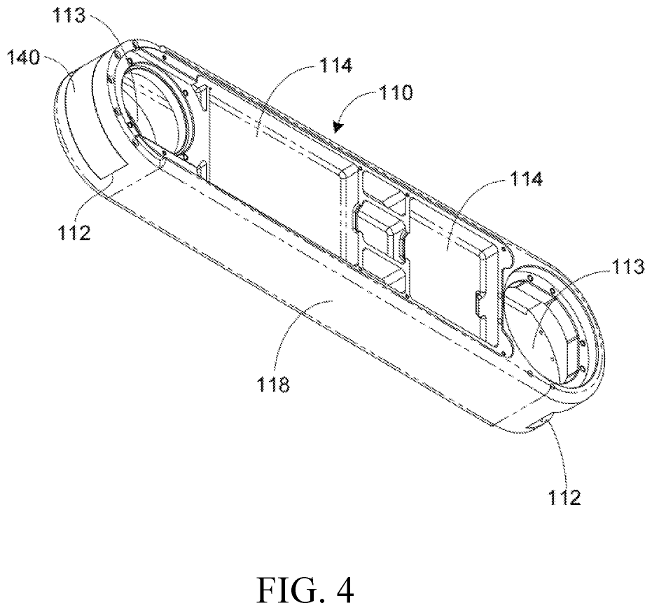

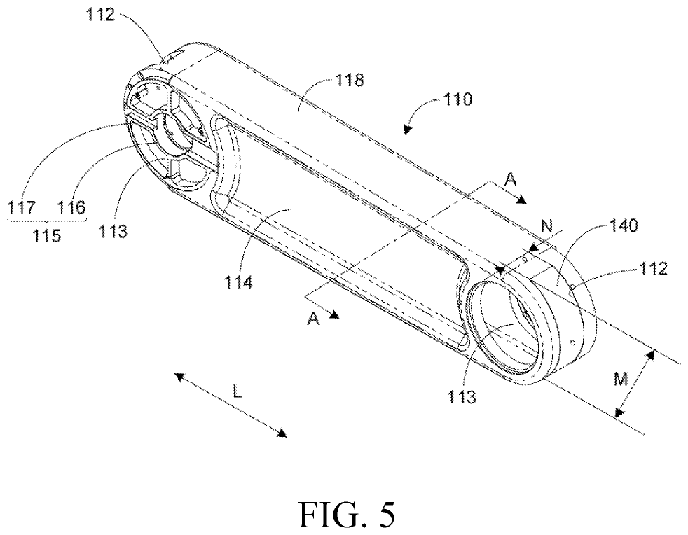

As shown in FIG. 4 and FIG. 5, the inner cavity 111 of the link 110 is in communication with two end openings 112 and two pairs of side openings 113. Preferably, each of the end openings 112 has a shape and a size which are adapted to a shape and a size of the cross section of the cavity 111 respectively. Specifically, the two end openings 112 are defined at both ends of the link 110 in the length direction L respectively and are generally configured as rectangular openings. Among the two pairs of side openings 113, one pair of side openings 113 is defined at one end of the link 110 in the length direction L and is oppositely arranged on both sides of the link 110 respectively, and the other pair of side openings 113 is defined at the other end of the link 110 in the length direction L and is oppositely arranged on both sides of the link 110 respectively. That is, the side openings 113 are disposed close to the end opening 112s, and the side openings 113 may be circular or in any other suitable shapes.

The robotic arm 100 further includes at least two flexible transmission assemblies and at least two pivot shafts 130 (see FIG. 3). In the inner cavity 111 of each link 110 is disposed at least one of the flexible transmission assemblies. In this embodiment, one flexible transmission assembly is disposed in the inner cavity 111 of each link 110, and the flexible transmission assemblies disposed in the respective inner cavities 111 of two adjacent links 110 are connected by a respective one of the pivot shafts 130. In this embodiment, the robotic arm 100 includes three flexible transmission assemblies as the robotic arm 100 includes three links 110 and each of which is provided with one flexible transmission assembly disposed in the respective inner cavity 111. It can be understood that the number of flexible transmission assemblies can be determined according to actual needs.

Specifically, the flexible transmission assembly includes a pair of pulleys 121 (see FIG. 3) and a flexible member (not shown) tensioned on the pair of pulleys 121. The pair of pulleys 121 are respectively disposed at the side openings 113 located at both ends of the inner cavity 111 of the link 110 and can enter into the inner cavity 111 through the end openings 112. In this embodiment, the flexible transmission assembly is a strap transmission assembly, the pulley 121 is a strap pulley, and the flexible member is a steel strap. In an embodiment not shown, the flexible transmission assembly may be a cable transmission assembly, and the flexible member may be a flexible cable.

The end openings 112 and/or the side openings 113 are configured to allow the flexible transmission assembly enter into the inner cavity, and to provide an access for a wrench to perform maintenance at the end such as tightening, thereby facilitating the mounting and tensioning of the flexible member on the pulleys and the maintenance of the transmission element such as the pulleys.

Preferably, as shown in FIG. 3, the end opening 112 has a length M (see FIG. 5) which is greater than an outer diameter of the pulley 121 and the end opening 112 has a width N (see FIG. 5) which is greater than a thickness of the pulley 121, which facilitates the mounting of the pulley 121 into the inner cavity 111 through the end opening 112. The respective one of the pivot shafts 130 is connected with the pulleys 121 in the respective inner cavities 111 of the two adjacent links 110 through the side openings 113. Specifically, the pulleys 121 in the respective inner cavities 111 of the two adjacent links 110 are sleeved on the same pivot shaft 130.

As shown in FIG. 3, the pivot shaft 130 includes an end cover portion 131 and a pivot portion 132 connected with the end cover portion 131. The end cover portion 131 has a shape of a substantially disk. The pivot portion 132 is disposed at the center of the end cover portion 131 and has a shape of a cylinder extending in a direction perpendicular to the end cover portion 131. The end cover portion 131 is disposed at the side opening 113 of one of two adjacent links 110 and is connected with the link 110, and specifically, the end cover portion 131 is disposed at the side opening 113 of the link 110 which is defined on a surface facing away from the adjacent link 110. The pulleys 121 in the inner cavities 111 of the two adjacent links 110 are sleeved on the pivot portion 132 of the same pivot shaft 130.

As shown in FIG. 3 and FIG. 5, the link 110 includes a main body 118 and a supporting portion 115 integrally formed with the main body 118. The inner cavity 111 is defined by the main body 118, and the supporting portion 115 is configured to support and/or limit the position of the flexible transmission assemblies. For example, the supporting portion 115 can support and limit the position of the pulleys 121 of the flexible transmission assembly in an axial direction.

Specifically, the supporting portion 115 is disposed in the side opening 113. The supporting portion 115 may be disposed in at least one of each pair of side openings 113. The supporting portion 115 includes a first supporting rib 116 extending in a circumferential direction and a second supporting rib 117 connected with the first supporting rib 116. The second supporting rib 117 extends outward from the first supporting rib 116 in a radial direction of the first supporting rib 116. The pivot portion 132 of the pivot shaft 130 passes through an inner hole defined by the first supporting rib 116, and the first supporting rib 116 can support and limit the position of the pivot portion 132.

As shown in FIG. 2 and FIG. 3, the robotic arm 100 may further include an end cover 140 for each of the end openings 112 and a side cover 150 for each of the side openings 113. The end cover 140 has a shape and a size which correspond to a shape and a size of the end opening 112 respectively, so as to be able to completely cover the end opening 112. The side cover 150 is disposed at a respective one of the side openings 113 and connected with the link 110. Specifically, the side cover 150 may be disposed at the side opening 113 of the link 110 which is defined on a surface facing away from adjacent link 110 connected with the end cover portion 131. The end openings 112 and the side openings 113 facilitate the operation and maintenance of the transmission member such as the pulleys 121.

The link 110 according to the present embodiment is one-piece. Specifically, the link 110 may be integrally formed (for example, formed by machining such as wire cutting), may be formed by drawing and then machining, or may be formed by casting.

In addition, the link 110 includes at least one recess 114 at an outer surface of the link 110. These recesses 114 is configured to accommodate at least one of wires, circuit boards, sensors, connecting flanges and other mechanical and electrical parts. FIG. 6 shows two recesses 114 which are located respectively on two opposing sides of the link 110 and at least one of which is configured to receive wires. The link 110 may further include a cover plate (not shown) for covering each recess 114, and the cover plate is connected to the link 110 and can cover the wires.

The link 110 of the present disclosure can be used not only in the above-mentioned flexible transmission system, but also in other types of belt transmission system or wire transmission system, etc., where there is a strong demand for the miniaturization of the cavity and high rigidity.

Unless otherwise defined, technical and scientific terms used herein have the same meaning as commonly understood by those skilled in the art of the present disclosure. The terminology used herein is for the purpose of describing a particular implementation only and is not intended to limit the present disclosure. A term such as “disposed” present herein means that one element is attached to the other element directly or by an intermediary piece. Features described herein in an embodiment may be applied to the embodiment alone or in combination with other features, unless the feature is not applicable in the other embodiment or stated otherwise.

The present disclosure has been described by the above-mentioned embodiments, but it should be understood that the above-mentioned embodiments are only for the purpose of illustration and description, and are not intended to limit the present disclosure to the scope of the described embodiments. It will be understood by those skilled in the art that various variations and modifications can be made according to the teachings of the present disclosure, which all fall within the scope of the claimed protection of the present disclosure.

What claimed is:

1. A link for a robotic arm, wherein the link defines an inner cavity extending in a length direction of the link, and
wherein at least a part of the inner cavity is seamlessly enclosed in a circumference perpendicular to the length direction of the link.

2. The link according to claim 1, wherein the link is one-piece.

3. The link according to claim 1, wherein the link is integrally formed.

4. The link according to claim 1, wherein the inner cavity extends straight from end to end of the link.

5. The link according to claim 1, wherein a cross section of the inner cavity has a shape of a rectangle, a circle or an ellipse.

6. The link according to claim 1, wherein the link includes at least one recess at an outer surface of the link.

7. The link according to claim 1, characterized in that the inner cavity is in communication with two end openings defined at both ends of the link in the length direction respectively, and each of the two end openings has a shape and a size which are adapted to a shape and a size of the cross section of the inner cavity.

8. The link according to claim 1, wherein the inner cavity is further in communication with two pairs of side openings, wherein a pair of side openings is defined at an end of the link in the length direction and is oppositely arranged on both sides of the link respectively, and the other pair of side openings is defined at the other end of the link in the length direction and is oppositely arranged on both sides of the link respectively.

9. The link according to claim 1, comprising a main body and a supporting portion integrally formed with the main body, the inner cavity is defined by the main body, and the supporting portion is configured to support a flexible transmission assembly and/or limit a position of the flexible transmission assembly.

10. The link according to claim 9, wherein the supporting portion comprises a first supporting rib extending in a circumferential direction and a second supporting rib connected with the first supporting rib and extending outward from the first supporting rib in a radial direction of the first supporting rib.

11. A robotic arm comprising at least one link, wherein the link defines an inner cavity extending in a length direction of the link, and
wherein at least a part of the inner cavity is seamlessly enclosed in a circumference perpendicular to the length direction of the link.

12. The robotic arm according to claim 11, comprising at least two links which are connected end to end and adjacent two of which are pivotably connected.

13. The robotic arm according to claim 12, further comprising at least two flexible transmission assemblies and at least one pivot shaft, in the inner cavity of each of the links is disposed at least one of the flexible transmission assemblies, and the flexible transmission assemblies disposed in the respective inner cavities of the adjacent two of the links are connected by a respective one of the at least one pivot shaft.

14. The robotic arm according to claim 13, wherein in the inner cavity of the each of the links is disposed one flexible transmission assembly which comprises a pair of pulleys and a flexible member tensioned on the pair of pulleys, and the respective one of the at least one pivot shaft is connected with the pulleys in the respective inner cavities of the adjacent two of the links.

15. The robotic arm according to claim 14, wherein the inner cavity is in communication with two end openings defined at both ends of the link in the length direction respectively.

16. The robotic arm according to claim 15, wherein the inner cavity is further in communication with two pairs of side openings, wherein a pair of side openings is defined at an end of the link in the length direction and is oppositely arranged on both sides of the link respectively, and the other pair of side openings is defined at the other end of the link in the length direction and is oppositely arranged on both sides of the link respectively.

17. The robotic arm according to claim 16, wherein the pair of pulleys are respectively disposed at the side openings at both ends of the inner cavity and enter into the inner cavity through the end openings, and the respective one of the at least one pivot shaft passes through the side openings to connects the pulleys in the respective inner cavities of the adjacent two of the links.

18. The robotic arm according to claim 15, further comprising an end cover for each of the end openings and a side cover for each of the side openings, and the side cover is disposed at a respective one of the side openings and connected with the link.

19. The robotic arm according to claim 11, wherein the link is one-piece, and the inner cavity extends straight from end to end of the link.

20. A surgical robot comprising at least one robotic arm, wherein the robotic arm comprises at least one link, the link defines an inner cavity extending in a length direction of the link, and at least a part of the inner cavity is seamlessly enclosed in a circumference perpendicular to the length direction of the link.
